(19) United States
(12) Patent Application Publication
(10) Pub. No.: US 2005/0208044 A1
(43) Pub. Date: Sep. 22, 2005

(54) Methods for the identification of compounds useful for the suppression of chronic neuropathic pain and compositions thereof

(76) Inventors: Jane Barclay, London (GB); Pamposh Ganju, London (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/506,551

(22) PCT Filed: Mar. 18, 2003
(86) PCT No.: PCT/EP03/02834

§ 371 Date: Apr. 26, 2005

Related U.S. Application Data
(60) Provisional application No. 60/365,487, filed on Mar. 19, 2002.

(51) Int. Cl. G01N 33/53; G01N 33/567; A61K 39/395; C07K 16/28

(52) U.S. Cl. 424/143.1; 435/7.2; 530/388.22

(57) ABSTRACT

The invention relates to a method of screening for a compound able to modulate the activity of an N-type voltage-dependent calcium channel consisting of the Caᵥ2.2 (α1B), α2δ1, β1 and γ4 subunits; wherein said compound may be in one embodiment e.g. an antibody or an antisense nucleotide sequence.

### FIELD OF THE INVENTION

This invention provides methods for identifying agents useful for the suppression of chronic neuropathic pain in mammals, in particular humans, by screening for the ability of a candidate compound to modulate the activity and/or expression of N-type voltage-dependent calcium channel (VDCC) consisting of the Cav2.2 (α1B), α2δ1, β1 and γ4 subunits. The invention also provides said agents, which are nucleic acids, ribozymes, and antibodies.

### DESCRIPTION OF THE RELATED ART

Compounds that regulate N-type VDCC activities, or are specific inhibitors of N-type VDCC are known. These include ω-conotoxin GVIA, SNX-111 (zinconotide), SNX-159, SNX 239, SNX-124 (Prado Wash. (2001) Braz J Med Biol Res 34: 449-461; Vanegas H, Schaible H (2000) Pain 85: 9-18). Gabapentin binds with high affinity to α2δ1, β1 and α2δ2 subunits and it may exert it's analgesic/anti-convulsant effect through modulation of VDCC currents, although this is controversial (Gong et al., J Memb Biol 184(1) 35-43 (2001); Sutton et al., Br J Pharmacol 135: 257-265 (2002)).

### DESCRIPTION OF THE INVENTION

“Antisense” refers to nucleotide sequences that are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter that permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, lie, Leu; Asp, Glu; Asn, Gln-I Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADIP ribosylation and the like. Embodiments include methylation of the N-terminal amino acid, phosphorylations of serines and threonines and modification of C-terminal glycines. Also included as variants are polynucleotides having SNP (see below) and encode for polypeptides with one or more amino acid exchange. Also included as variants are polynucleotides which are so called splice variants (see below) and therefore encode altered polypeptides.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length.

Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25: 389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448, 1988, available as part of the Wisconsin Sequence Analysis Package).

Preferably, the BLOSUM62 amino acid substitution matrix (Henikoff S and Henikoff J G, Proc. Nat. Acad. Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5-25 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of nucleotide or amino acid differences and the Identity Index may be expressed in the following equation:
nₐ≦xₐ−(xₐ•I)

in which:
nₐ is the number of nucleotide or amino acid differences,
xₐ is the total number of nucleotides or amino acids for any given sequence (e.g. 596 for SEQ ID NO: 1),
I is the Identity Index,
• is the symbol for the multiplication operator, and in which any non-integer product of xₐ and I is rounded down to the nearest integer prior to subtracting it from xₐ.
“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotide or polypeptide that within the same species which is functionally similar.

“Fusion protein” refers to a protein encoded by two, unrelated, fused genes or fragments thereof. Examples have been disclosed in U.S. Pat. Nos. 5,541,087, 5,726,044. Employing an immunoglobulin Fc region as a part of a fusion protein is advantageous for performing the functional expression of the protein of interest, to improve pharmacokinetic properties of such a fusion protein when used for therapy and to generate a dimeric fusion protein. The fusion protein DNA construct may comprise in 5′ to 3′ direction, a secretion cassette, i.e. a signal sequence that triggers export from a mammalian cell, DNA encoding an immunoglobulin Fc region fragment, as a fusion partner, and a DNA encoding a protein of interest or fragments thereof. In some uses it would be desirable to be able to alter the intrinsic functional properties (complement binding, Fc-Receptor binding) by mutating the functional Fc sides while leaving the rest of the fusion protein untouched or delete the Fc part completely after expression.

The present invention provides a method for screening compounds that inhibit, modulate, down-regulate or immobilize (in the cell) one or more Pain VDCC. It has been found that not only the Caᵥ2.2 (α1B), α2δ1, β1 and γ4 subunits, but also the β1 and γ4 subunits of the VDCC are up-regulated in animal models of chronic neuropathic pain (e.g. Seltzer et al., (1990) Pain 43: 205-218, Chronic Construction Injury model (G. J and Xie, Y. K. Pain (1988) 33: 87-107), or Chung model (Kim, S. O. and Chung, J. M. Pain (1992) 50: 355-363)), e.g. it was found that its messenger RNA is expressed in DRGs of those animals (see Table 2 comparing up-regulation of different subunits in different neuropathic pain models). Pain VDCC consisting of Caᵥ2.2 (α1B), α2δ1, β1 and γ4 subunits or variants (including e.g. splice variants, SNPs) of said subunits of the VDCC can be used to screen drugs for the treatment of chronic neuropathic pain states associated with diseases including but not limited to the following: osteroarthritis, rheumatoid arthritis, cancer, diabetes, mechanical nerve injuries, postherpetic neuralgia, chronic lower back pain, abdominal pain and spinal stenosis. Such variants have more than 80% identity, more preferentially more than 85, 86, 87, 88, 89, 90, 91, 92, 93, 94, 95, 96, 97, 98, or 99% identity. Levels of expression of the Pain VDCC or subunits thereof can be assayed from a biological sample, e.g., tissue (e.g. Dorsal Root Ganglion, DRG) sections, cell lysate, tissue lysate or white blood cell lysate, by any known methods, including in situ hybridization, quantitative PCR, immunoassays and electrophoresis assays (see also example 1). Test compounds which can be used in the present invention can be obtained using any of the numerous approaches in combinatorial library methods known in the art, including: biological libraries; spatially addressable parallel solid phase or solution phase libraries; synthetic library methods requiring deconvolution; the ‘one- bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12: 145). Examples of methods for the synthesis of molecular libraries can be found in the art, for example in DeWitt et al. (1993) Proc. Natl. Acad. Sci. U.S.A. 90: 6909; Erb et al. (1994) Proc. Natl. Acad. Sci. USA 91: 11422; Zuckermann et al. (1994). J. Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and in Gallop et al. (1994) J. Med. Chem. 37: 1233. Libraries of compounds may be presented in solution (e.g., Houghten (1992) Biotechniques 13: 412-421), or on beads (Lam (1991) Nature 354: 82-84), chips (Fodor (1993) Nature 364: 555-556), bacteria (Ladner U.S. Pat. No. 5,223,409), spores (Ladner U.S. Pat. No. '409), plasmids (Cull et al. (1992) Proc Natl Acad Sci USA 89: 1865-1869) or on phage (Scott and Smith (1990) Science 249: 386-390); (Devlin (1990) Science 249: 404-406); (Cwirla et al. (1990) Proc. Natl. Acad. Sci. 87: 6378-6382); (Felici (1991) J. Mol. Biol. 222: 301-310); (Ladner supra.). The currently available VDCC blockers have unacceptable side effects which are due, at least in part, to non-specific channel blocking (other ion channel, other VDCCs). The Pain VDCC is expected to exist in a very limited number of tissues, and it may be the case that splice variants result in total tissue specificity. The Caᵥ2.2 subunit is only expressed in neuronal tissue and splice variants distinguish brain and peripheral channels. The β2δ1 subunit is widely expressed, but at least 5 alternative splice variants have been identified. The β1 subunit is predominantly expressed in skeletal muscle, but two brain specific isoforms exist. The γ4 subunit is only expressed in neuronal tissue. It is expected that the same combination of splice variants won't exist outside of DRG neuronal sub-populations which should reduce side effects, but would mean that the efficacy of treatment could not be monitored by, for example, by blood sampling. In one embodiment, a screening assay comprises contacting a recombinant cell, which expresses the Pain VDCC, with a test compound, e.g. from above-mentioned libraries. One further determines then the ability of the test compound to modulate (by e.g. stimulating or inhibiting the Pain VDCC or by up/down-regulation of the expression of Pain VDCC or by immobilization or mobilization of Pain VDCC from intracellular pools) VDCC activity. Compounds identified by the above-described method can further be used and its activity confirmed in an appropriate animal model, e.g. the animal models described above (Seltzer model, Chronic Construction Injury model, or Chung model). In another embodiment of this invention, it relates to the monitoring of effects during clinical trials to evaluate a treatment, both in basic drug screening and in clinical trials. For example, the effectiveness of a compound determined by a screening assay as described herein to inhibit the Pain VDCC activity can be monitored in clinical trials of subjects exhibiting chronic neuropathic pain. In a preferred embodiment, the present invention provides a method for evaluating, e.g., monitoring, the effectiveness of treatment of a subject with a compound (e.g., peptidomimetic, protein, peptide, nucleic acid, small molecule, or other drug candidate identified by the screening assays described herein) comprising the steps of (i) evaluating pre-administration levels of Pain VDCC activity in a subject prior to administration of a compound using detection methods known in the art, (ii) administering a compound to the subject; (iii) evaluating the level of Pain VDCC activity post-administration of the compound; and (iv) comparing the levels prior to and subsequent to the administration of the compound. According to such an embodiment, the levels of Pain VDCC of the subject may be used as an indicator of the effectiveness of a compound.

The invention further provides substances that inhibit the expression of β1 or γ4 subunits of the Pain VDCC at the nucleic acid level. Such molecules include ribozymes, antisense oligonucleotides, triple helix DNA, RNA aptamers and/or double stranded RNA directed to an appropriate nucleotide sequence of β1 or γ4 nucleic acid. These inhibitory molecules may be created using conventional techniques by one of skill in the art without undue burden or experimentation. For example, modifications (e.g. inhibition) of gene expression can be obtained by designing antisense molecules, DNA or RNA, to the control regions of the genes encoding the polypeptides discussed herein, i.e. to promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art. Typically, oligonucleotides between about 5 and 50 nucleotides in length are used. More preferably, oligonucleotides between about 5 and 35 nucleotides are used. Even more preferably, oligonucleotides about 20 nucleotides in length are used. An antisense oligonucleotide can be constructed using chemical synthesis procedures known in the art. An oligonucleotide can be chemically synthesized using naturally occurring nucleotides or variously modified nucleotides designed to increase the biological stability of the oligonucleotide or to increase the physical stability of the duplex formed between the antisense and sense nucleic acids. For example, the use of phosphorothioate, methyl phosphonate and ethyl phosphotriester antisense oligonucleotides (reviewed in Stein, C. A. and Cheng Y-C. (1993) Science 2261: 1004-1012) is within the scope of the invention. Additionally, acridine substituted nucleotides can be incorporated into the antisense oligonucleotides used in the present invention. A preferred antisense nucleic acid of the invention is an antisense to a coding or regulatory region of one of the subunit genes of the Pain VDCC (e.g. β1 or γ4 gene). As contemplated herein, antisense oligonucleotides, triple helix DNA, RNA aptamers, ribozymes and double stranded RNA are directed to a nucleic acid sequence of β1 or γ4 such that the chosen nucleotide sequence of β1 or γ4 will produce gene-specific inhibition of β1 or γ4 gene expression. For example, knowledge of the β1 or γ4 nucleotide sequence may be used to design an antisense molecule that gives strongest hybridization to the mRNA. Similarly, ribozymes can be synthesized to recognize specific nucleotide sequences of β1 or γ4 and cleave it (Cech. J. Amer. Med Assn. 260: 3030 (1988). Techniques for the design of such molecules for use in targeted inhibition of gene expression is well known to one of skill in the art. Gene specific inhibition of gene expression may also be achieved using conventional double stranded RNA technologies. A description of such technology may be found in WO 99/32619 which is hereby incorporated by reference in its entirety. Antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues. Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well known in the art. It is contemplated herein that one can inhibit the function and/or expression of a gene for a related regulatory protein or protein modified by β1 or γ4 as a way to treat chronic pain by designing, for example, antibodies to these proteins and/or designing inhibitory antisense oligonucleotides, triple helix DNA, ribozymes and RNA aptamers targeted to the genes for such proteins according to conventional methods. Pharmaceutical compositions comprising such inhibitory substances for the treatment of chronic pain are also contemplated. Antisense oligonucleotides, or an antisense recombinant expression vector can be designed based upon the known nucleotide sequence of one of the subunit cDNAs of the Pain VDCC (e.g. β1 or γ4 cDNAs) known in the art. The nucleotide sequence of a human β1 subunit cDNA is available from Genbank™ (Accession Number NM_{—}000723), whereas the nucleotide sequence of a human γ4 subunit cDNA is available from Genbank™ (Accession Number NM_{—}014405). In addition, the nucleotide sequence of a rat β1 subunit cDNA is available from Genbank™ (Accession Number NM_{—}017346), and the nucleotide sequence of a rat γ4 subunit cDNA is available from Genbank™ (Accession Number AF361341). To inhibit the activity of the Pain VDCC in a cell from another species, antisense oligonucleotides are designed which are complimentary to nucleotide sequences that are conserved among the different subunit genes in different species (e.g., based upon comparison of the known subunit sequences, including the human and murine sequences, to identify conserved regions). Antisense oligonucleotides can be used to inhibit the activity of Pain VDCC in a cell by genetic therapy and/or exogenously administering them to a subject at an amount and for a time period sufficient to inhibit transcription of a gene of one the subunits of Pain VDCC (e.g. β1 and γ4 gene) or translation of the mRNA of one the subunits of Pain VDCC (e.g. β1 or γ4 gene) in the cell. In one embodiment, an antisense nucleic acid can be produced biologically using an expression vector into which a nucleic acid has been sub-cloned in an antisense orientation (i.e., nucleic acid transcribed from the inserted sequence will be in an antisense orientation relative to a target nucleic acid of interest). The antisense expression vector is introduced into cells, for example, in the form of a recombinant plasmid, phagemid or attenuated virus in which antisense nucleic acids are produced under the control of a high efficiency regulatory region of the vector, the activity of which can be determined by the cell type into which the vector is introduced. Preferably, the recombinant expression vector is a recombinant viral vector, such as a retroviral, adenoviral or adeno-associated viral vector. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM that are well known to those skilled in the art. Examples of suitable packaging virus lines include WCrip, yCre, y2 and yAm. Adenoviral vectors are described in Berkner et al. (1988) BioTechniques 6: 616; Rosenfeld et al. (1991) Science 252: 431-434; and Rosenfeld et al. (1992) Cell 68: 143-155. Suitable adenoviral vectors derived from the adenovirus strain Ad type 5 d1324 or other strains of adenovirus (e.g., Ad2, Ad3, Adz etc.) are well known to those skilled in the art. Adeno-associated vectors (MV) are reviewed in Muzyczka et al. Curr. Topics in Micro. and Immunol. (1992) 158: 97-129). An example of a suitable MV vector is described in Tratschin et al. (1985) Mol. Cell. Biol. 5: 3251-3260. A recombinant expression vector containing a nucleic acid in an antisense orientation is introduced into a cell to generate antisense nucleic acids in the cell to thereby inhibit the activity of the Pain VDCC in the cell. The vector can be introduced into a cell by a conventional method for introducing nucleic acid into a cell. When a viral vector is used, the cell can be infected with the vector by standard techniques. Cells can be infected in vitro or in vivo. When a non-viral vector, e.g., a plasmid, is used, the vector can be introduced into the cell by, for example, calcium phosphate precipitation, DEAE-dextran transfection, electroporation or other suitable method for transfection of the cell.

Additionally, the present invention relates to a method for identifying a compound useful for the treatment of chronic neuropathic pain, the method comprising: a) contacting a ligand of an N-type voltage-dependent calcium channel consisting of the Caᵥ2.2 (α1B), α2δ1, β1 and γ4 subunits with an N-type voltage-dependent calcium channel consisting of the Caᵥ2.2 (α1B), α2β1, β1 and γ4 subunits in the presence and absence of a test compound; and b) determining whether the test compound alters the binding of the ligand to the N-type voltage-dependent calcium channel consisting of the Caᵥ2.2 (α1B), α2δ1, β1 and γ4 subunits to an N-type voltage-dependent calcium channel consisting of the Caᵥ2.2 (α1B), α2δ1, β1 and γ4 subunits. Optionally, said method further comprises the steps of: c) adding a compound identified that alters binding of the ligand to the N-type voltage-dependent calcium channel consisting of the Caᵥ2.2 (α1B), α2δ1, β1 and γ4 subunits to an N-type voltage-dependent calcium channel consisting of the Caᵥ2.2 (α1B), α2δ1, β1 and γ4 subunits in step (b); d) determining whether the compound alleviates chronic neuropathic pain; and e) identifying a compound that alleviates chronic neuropathic pain in step (d) as a compound useful for the treatment of chronic neuropathic pain.

In yet another embodiment, a nucleic acid used to inhibit the Pain VDCC activity in a cell is a ribozyme which is capable of cleaving a single-stranded nucleic acid encoding one of the subunits of the Pain VDCC, such as an mRNA transcript. A catalytic RNA (ribozyme) having ribonuclease activity can be designed which has specificity for an mRNA encoding one of the subunits of the Pain VDCC. For example, a derivative of a Tetrahymena L-19 IVS RNA can be constructed in which the base sequence of the active site is complementary to the base sequence to be cleaved in a pain VDCC mRNA. See for example Cech et al. U.S. Pat. No. 4,987,071; Cech et al. U.S. Pat. No. 5,116,742 for descriptions of designing ribozymes. Alternatively, a RNA of one of the subunits of the Pain VDCC (e.g. β1 or γ₁ subunit) can be used to select a catalytic RNA having specific ribonuclease activity against RNA of one of the subunits of the Pain VDCC from a pool of RNA molecules. See for example, Bartel, D. and Szostak, J. W. Science 261: 1411-1418 (1993) for a description of selecting ribozymes. A ribozyme can be introduced into a cell by constructing a recombinant expression vector (e.g., a viral vector as discussed above) containing nucleic acid which, when transcribed, produces the ribozyme (i.e., DNA encoding the ribozyme is cloned into a recombinant expression vector by conventional techniques).

In certain additional preferred embodiments of the invention there is provided an antibody or a fragment thereof which specifically binds to the Pain VDCC or to the γ4 subunit. Antibodies are commercially available to the subunits Caᵥ2.2 (α1B), α2δ1, β1. Described herein are methods for the production of antibodies capable of specifically recognizing one or more differentially expressed gene epitopes of the Pain VDCC. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-id) antibodies, and epitope-binding fragments of any of the above. Such antibodies may be used, for example, in the detection of a fingerprint, Pain VDCC gene in a biological sample, or, alternatively, as a method for the inhibition of abnormal Pain VDCC activity. Thus, such antibodies may be utilized for the suppression of chronic neuropathic pain in human and veterinary patients. Chronic neuropathic pain results from damage to nerves by trauma, by diseases such as diabetes, herpes zoster, or late-stage cancer, or by chemical injury (e.g some anti-HIV drugs). It may also develop after amputation (including mastectomy), and is involved in some low-back pain (Portenoy R K. Neuropathic pain. In: Portenoy R K, Kanner R M, (Eds). Pain Management: Theory and Practice. Philadelphia: F A Davis, 1996, pp 83-125). For the production of antibodies to a differentially expressed gene, various host animals may be immunized by injection with a differentially expressed gene protein, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with differentially expressed gene product supplemented with adjuvants as also described above. Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (e.g. U.S. Pat. No. 4,376,110), the human B-cell hybridoma technique (e.g. Cole et al., 1983, Proc. Natl. Acad. Sci. USA 80: 2026-2030), and the EBV-hybridoma technique (e.g. Cole et al., 1985, Monoclonal Antibodies And Cancer Therapy, Alan R. Liss, Inc., pp. 77-96). Such antibodies may be of any immunoglobulin class Including IgG, IgM, IgE, IgA, IgD and any subclass thereof. The hybridoma producing the mAb of this invention may be cultivated in vitro or in vivo. Production of high titers of mAbs in vivo makes this the presently preferred method of production. In addition, techniques developed for the production of “chimeric antibodies” (e.g. Morrison et al., 1984, Proc. Natl. Acad. Sci., 81: 6851-6855) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (e.g. U.S. Pat. No. 4,946,778) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed e.g. in U.S. Pat. No. 5,770,429, the disclosures of which are incorporated by reference herein in their entirety.

Antibody fragments which recognize specific epitopes may be generated by known techniques. Preferred antibodies of the present invention have at least one activity of Pain VDCC.

Additionally, the present invention pertains to the use of an antibody as disclosed herein or a compound that binds to the β1 and γ4 subunits of an N-type voltage-dependent calcium channel consisting of the Caᵥ2.2 (α1B), α2δ1, β1 and γ4 subunits in medicine and, in particular, for the manufacture of a medicament for the treatment of chronic neuropathic pain.

A compound that binds to the β1 and γ4 subunits of an N-type voltage-dependent calcium channel consisting of the Caᵥ2.2 (α1B), α2δ1, β1 and γ4 subunits can be applied in vitro in the form of solutions, e.g. preferably aqueous solutions or suspensions, and in vivo either enterally or parenterally, advantageously orally, e.g. as a suspension or in aqueous solution, or as a solid capsule or tablet formulation.

Hence, the present invention furthermore provides
(a) a method for the treatment of chronic neuropathic pain which comprises administering an effective amount of a compound that binds to the β1 and γ4 subunits of an N-type voltage-dependent calcium channel consisting of the Caᵥ2.2 (α1B), α2δ1, β1 and γ4 subunits to a patient in need of such treatment; and
   (b) a pharmaceutical composition for the treatment of chronic neuropathic pain which comprises a compound that binds to the β1 and γ4 subunits of an N-type voltage-dependent calcium channel consisting of the Caᵥ2.2 (α1B), α2δ1, β1 and γ4 subunits, and a pharmaceutically acceptable carrier or diluent.

The following examples further illustrate the present invention, and the examples are provided for illustration purposes and are not intended to be limiting the invention.

### EXAMPLE 1

### Expression Profiling of VDCC subunits in Naïve and Animal Models of Neuropathic Hyperalgesia

(1) VDCC subunit gene expression in naïve rat DRG: Initially, all the primer sets described in Table 1 are used to test naïve DRG total RNA for gene expression. Total RNA samples are prepared from dissected DRG tissues from ten naïve rats using Tri Reagent (Sigma) as per manufacturers instructions. Two μg of total RNA is treated with 0.2 units of RNAse-free Dnase I (Roche Diagnostics) at 37° C. for 5 minutes and purified using a RNA-easy column (Qiagen) as per manufacturers instructions. The concentration of RNA is determined by OD. 2 μg of DNAse treated RNA is transcribed into cDNA using First strand cDNA synthesis kit (Amersham Pharmacia) in a 66 μl volume (2× scaled reaction) as per manufacturers instructions. The cDNA samples are diluted to 25 ng/μl RNA equivalent cDNA and 50 ng used in a standard RT-PCR reaction using Qiagen Hot Start Taq Polymerase (Qiagen) with annealing temperatures of 50-60° C.

| | | | |
|---|---|---|---|
| | | | Pro- |
| | Accession | Primers used for PCR | duct |
| Gene | number | (Sequence 5′→3′) | (bp) |

| | | | |
|---|---|---|---|
| α2δ1 | R: NM_012919 | CAG ACA TAC TCC AGA TTG | 529 |
| | (SEQ ID NO: 6) | GC | |
| | H: XM_004914 | (SEQ ID NO: 12) | |
| | (SEQ ID NO: 7) | TAG TGT CTG CTG CCA GAT | |
| | | AC | |
| | | (SEQ ID NO: 13) | |
| α2δ2 | M: AF247139 | CAG CCT GGA TAA CCA TGG | 604 |
| | | TT | |
| | | (SEQ ID NO: 14) | |
| | | AGC TGT GAT AGA TGA GAC | |
| | | CA | |
| | | (SEQ ID NO: 15) | |
| α2δ3 | M: NM_009785 | AAC AAG GAG AGC AGT GAC | 521 |
| | | AG | |
| | | (SEQ ID NO: 16) | |
| | | AGA AGA GAC TCG AAA CCA | |
| | | GG | |
| | | (SEQ ID NO: 17) | |
| β1 | R: NM_017346 | ACA TGA TGC AGA AGG CGT | 1025 |
| | (SEQ ID NO: 1) | TG | |
| | H: NM_000723 | (SEQ ID NO: 18) | |
| | (SEQ ID NO: 2) | CCA GCT CAT TCT TAT TGC | |
| | | GC | |
| | | (SEQ ID NO: 19) | |
| β2 | R: M80545 | CTT ACG ATG TGG TAC CAT | 1040 |
| | | CC | |
| | | (SEQ ID NO: 20) | |
| | | TGC ACT CAT TGT GGT CTT | |
| | | GG | |
| | | (SEQ ID NO: 21) | |
| β3 | R: NM_012828 | ACA GAC ATG ATG CAG AAG | 952 |
| | | GC | |
| | | (SEQ ID NO: 22) | |
| | | ATC TAA CTC CAA CCT GCC | |
| | | TG | |
| | | (SEQ ID NO: 23) | |
| β4 | R: L02315 | TGA TTG GTG GAT AGG AAG | 1072 |
| | | GC | |
| | | (SEQ ID NO: 24) | |
| | | AAT CTT CTT CCA CCA GAG | |
| | | GG | |
| | | (SEQ ID NO: 25) | |
| Ca_{ν}2. | R: NM_012918 | CAG GAG ATG TTC CAG AAG | 448 |
| 1 | | AC | |
| α1A | | (SEQ ID NO: 26) | |
| | | TGG TCA TGC TCA GAT CTG | |
| | | TC | |
| | | (SEQ ID NO: 27) | |
| Ca_{ν}2. | R: AF055477 | ACC TAC AAG ACG GCC AAT | 624 |
| 2α1B | (SEQ ID NO: 8) | TC | |
| | H: AF055477 | (SEQ ID NO: 28) | |
| | (SEQ ID NO: 9) | GAG AGG ACC ATA TCC CTC | |
| | | TA | |
| | | (SEQ ID NO: 29) | |
| Ca_{ν}1. | R: M67515 | GAC ATC TCT CAG AAG ACA | 400 |
| 2α1C | | GC | |
| | | (SEQ ID NO: 30) | |
| | | ACA GGT TGC TGA CAT AGG | |
| | | AC | |
| | | (SEQ ID NO: 31) | |
| Ca_{ν}1. | R: NM_017298 | ACC TTA TGC AGC AGC AGA | 549 |
| 3α1D | | TC | |
| | | (SEQ ID NO: 32) | |
| | | CAA AGT CCT GGA GCT CAT | |
| | | AG | |
| | | (SEQ ID NO: 33) | |
| Ca_{ν}2. | R: L15453 | TTA CCG AGT CTT CCA ACT | 359 |
| 3α1E | | CC | |
| | | (SEQ ID NO: 34) | |
| | | GTG AGA GAA GAG CAT GCA | |
| | | TC | |
| | | (SEQ ID NO: 35) | |
| Ca_{ν}1. | H: NM_005183 | TTC ACC ATC CAG TGT CTG | 375 |
| 4α1F | M: NM_019582 | CA | |
| | | (SEQ ID NO: 36) | |
| | | CAT CTG CAA TCT CCT GCT | |
| | | TG | |
| | | (SEQ ID NO: 37) | |
| Ca_{ν}3. | R: AF027984 | AGA GAC CAG AAG CAG CTT | 495 |
| 1α1G | | AG | |
| | | (SEQ ID NO: 38) | |
| | | CAT GTC TGT TGG GTC AGA | |
| | | AG | |
| | | (SEQ ID NO: 39) | |
| Ca_{ν}3. | M: AF226868 | GGA ACA ACA ACC TGA CCT | 431 |
| 2α1H | H: AF051946 | TC | |
| | | (SEQ ID NO: 40) | |
| | | GAT CAT GAA GAA GGA GCC | |
| | | CA | |
| | | (SEQ ID NO: 41) | |
| Ca_{ν}3. | R: AF08627 | ACA GGC GAT AAC TGG AAT | 584 |
| 3α1I | | GG | |
| | | (SEQ ID NO: 42) | |
| | | GAT GAA GAC CTG TCT CTT | |
| | | GC | |
| | | (SEQ ID NO: 43) | |
| γ1 | R: NM_019255 | TGA CCC TCT TCT TCA TCC | 643 |
| | | TG | |
| | | (SEQ ID NO: 44) | |
| | | GAT CTA GTG CTC TGA CTC | |
| | | AG | |
| | | (SEQ ID NO: 45) | |
| γ2 | M: NM_007583 | GCT GTT TGA TCG AGG TGT | 808 |
| | H: NM_006078 | TC | |
| | | (SEQ ID NO: 46) | |
| | | TGT ACA TGG AGA TCT CCG | |
| | | TG | |
| | | (SEQ ID NO: 47) | |
| γ3 | M: NM_019430 | ATG AGG ATG TGT GAC AGA | 776 |
| | H: NM_006539 | GG | |
| | | (SEQ ID NO: 48) | |
| | | TGT GGA AGC CTT TGC TGA | |
| | | TG | |
| | | (SEQ ID NO: 49) | |
| γ4 | M: NM_019431 | ACC AAC CTG ACC ATG GAC | 583 |
| | (SEQ ID NO: 5) | GAC | |
| | H: NM_014405 | (SEQ ID NO: 50) | |
| | (SEQ ID NO: 4) | TAC CTG TAG CTC GGC ATC | |
| | R: AF361341 | CTG | |
| | SEQ ID NO: 3) | (SEQ ID NO: 51) | |
| γ5 | H: NM_014404 | TGA GCA GTG TCT TTG CTG | 562 |
| | | TC | |
| | | (SEQ ID NO: 52) | |
| | | ATC CAT ACT TAG GGT GAC | |
| | | GG | |
| | | (SEQ ID NO: 53) | |
| γ5* | M: NM_019432 | AAG CTG TTG GGC CTT AAG | 597 |
| | | AG | |
| | | (SEQ ID NO: 54) | |
| | | AAG TCC CAT CAT AAC CTC | |
| | | GC | |
| | | (SEQ ID NO: 55) | |
| γ6 | H: AF288386 | GGG TGG AGC TCA ACA CCT | 230 |
| | | AC | 859 |
| | | (SEQ ID NO: 56) | |
| | | GCC CAG GGA CCA GGA GTA | |
| | | (SEQ ID NO: 57) | |
| | | ATA CAG AGG CAC CCC AAG | |
| | | G | |
| | | (SEQ ID NO: 58) | |
| γ7 | H: AF288387 | CGG TCA GCA CTG ACT ACT | 625 |
| | | GG | |
| | | (SEQ ID NO: 59) | |
| | | ATG ACC TCG TCG TTG ATG | |
| | | C | |
| | | (SEQ ID NO: 60) | |
| γ8 | H: AF288388 | ATG ACC ATC GCC ATC ATC | 900 |
| | | AGC | |
| | | (SEQ ID NO: 61) | |
| | | CGC CCA GAA TGA TGT TCC | |
| | | (SEQ ID NO: 62) | |

Table 1. PCR primers designed to VDCC subunit genes. Accession numbers prefixed with R, M or H correspond to rat, mouse or human cDNA sequences respectively. PCR primers are designed to these sequences.
*Two γ5 subunits, with different sequences, have been deposited in Genbank. The Ca_{ν}1.1 (α1S) gene is not analysed since this is thought to be skeletal muscle specific, however subsequent reports have reported low levels of neuronal expression of this gene.

Genes are regarded as being expressed in naïve rat DRG if products of the correct size are clearly visible after standard agarose gel electrophoresis. The primers for the β, γ and α2δ and Caᵥ2.2 (α1B) subunits are then optimised for quantitative RT-PCR analysis using the LightCycler Technology (Roche Diagnostics). Naïve rat DRG cDNA is amplified using conditions suggested with the LightCycler Faststart DNA Master SYBR Green I kit (Roche Diagnostics). Annealing temperatures are determined empirically. Once optimized, the conditions are used to screen a panel of cDNAs from neuropathic pain models and controls as described below. Four animal models of neuropathic pain are used:

1.) Seltzer model: In the Seltzer model (Seltzer et al., (1990) Pain 43: 205-218) rats are anaesthetized and a small incision made mid-way up one thigh (usually the left) to expose the sciatic nerve. The nerve is carefully cleared of surrounding connective tissue at a site near the trochanter just distal to the point at which the posterior biceps semitendinosus nerve branches off the common sciatic nerve. A silk 7-0 silk suture is inserted into the nerve with a ⅜ curve, reverved-cutting mini-needle, and tightly ligated so that the dorsal ⅓ to ½ of the nerve thickness is held within the ligature. The muscle and skin are closed with sutures and clips and the wound dusted with antibiotic powder. In sham animals the sciatic nerve is exposed but not ligated and the wound closed as in non-sham animals.

2.) Chronic Constriction Injury (CCl) model: In the CCl model (Bennett, G. J. and Xie, Y. K. Pain (1988)_{—}33: 87-107) rats are anaesthetized and a small incision is made mid-way up one thigh (usually the left) to expose the sciatic nerve. The nerve is cleared of surrounding connective tissue and four ligatures of 4/0 chromic gut are loosely tied around the nerve with approximately 1 mm between each, so that the ligatures just barely constrict the surface of the nerve. The wound is closed with sutures and clips as described above. In sham animals the sciatic nerve is exposed but not ligated and the wound closed as in non-sham animals.

3.) Axotomy model: The axotomy model involves complete cut and ligation of the sciatic nerve. The nerve endings form neuromas but there is no behavioural correlate in this model as the nerve is not allowed to regenerate, and the foot is permanently denervated (Wall et al., (1979) Pain: 7: 103-113).

4.) Chung model: In contrast to the Seltzer and CCl models that involve damage to peripheral nerves, the Chung model involves ligation of the spinal nerve (Kim, S. O. and Chung, J. M. Pain (1992): 50: 355-363). In this model, rats are anesthetized and placed in a prone position and an incision is made to the left of the spine at the L4-S2 level. A deep dissection through the paraspinal muscles and separation of the muscles from the spinal processes at the L4-S2 level will reveal part of the sciatic nerve as it branches to form the L4, L5 and L6 spinal nerves. The L6 transverse process is carefully removed with a small rongeur enabling visualisation of these spinal nerves. The L5 spinal nerve is isolated and tightly ligated with 7-0 silk suture. The wound is closed with a single muscle suture (6-0) silk and one or two skin closure clips and dusted with antibiotic powder. In sham animals the L5 nerve is exposed as before but not ligated and the wound closed as before.

Validation of the pain models: In all chronic pain models mechanical hyperalgesia is assessed by measuring paw withdrawal thresholds of both hindpaws to an increasing pressure stimulus using an Analgesymeter (Ugo-Baile, Milan). Mechanical allodynia is assessed by measuring withdrawal thresholds to non-noxious mechanical stimuli applied with von Frey hairs to the plantar surface of both hindpaws. Thermal hyperalgesia is assessed by measuring withdrawal latencies to a noxious thermal stimulus applied to the underside of each hindpaw. With all models, mechanical hyperalgesia and allodynia and thermal hyperalgesia develop within 1-3 days following surgery and persist for at least 50 days. In the experiments disclosed herein, male Wistar rats are employed in the pain models described above. Rats weigh approximately 120-140 grams at the time of surgery. All surgery is performed under enflurane/O2 inhalation of anaesthesia. In all cases, the wound is closed after the procedure and the animal allowed to recover. In all but the axotomy model, a marked mechanical and thermal hyperalgesia develops in which there is a lowering of pain threshold and an enhanced reflex withdrawal response of the hind-paw to touch, pressure of thermal stimuli. After surgery, the animals also exhibit characteristic changes to the affected paw. In the majority of animals the toes of the affected hindpaw are held together and the foot turned slightly to one side; in some rats the toes are also curled under. The gait of the ligated rat varied, but limping is uncommon. Some rats are seen to raise the affected hindpaw from the cage floor and to demonstrate an unusual rigid extension of the hindlimb when held. The rats tend to be very sensitive to touch and may vocalise. Otherwise the general health and condition of the rats is good.

RNA Extraction from Dorsal root ganglia taken from animal models: L4 and L5 Dorsal Root Ganglia, ipsilateral to the nerve injury, are dissected at days 14, 21, 28 and 50 after surgery from rat models of neuropathic pain (described above). Contralateral L4 and L5 DRGs are taken for control samples as are DRGs from sham operated animals. Total RNA samples are prepared from the dissected DRG tissues using Tri Reagent (Sigma) as per manufacturers instructions. Two μg of total RNA is treated with 0.2 units of RNAse-free Dnase I (Roche Diagnostics) at 37° C. for 5 minutes and purified using a RNA-easy column (Qiagen) as per manufacturers instructions. The concentration of RNA is determined by OD. 2 μg of DNAse treated RNA is transcribed into cDNA using First strand cDNA synthesis kit (Amersham Pharmacia) in a 66 μl volume (2× scaled reaction) as per manufacturers instructions. The cDNA samples are diluted to 12.5 ng/μl RNA equivalent cDNA for LightCycler analysis. A standard curve of known concentrations of RNA equivalent cDNA from naïve rat DRG (50-1 ng) is run alongside the panel of samples. The relative cross-over points in the linear range of amplification are determined and used to quantify the sample message levels. Each run is performed in triplicate and all values averaged and standard error determined. Levels of b-actin mRNA are also determined using commercially available primers (Ambion, catalog number 1720) and the LightCycler Faststart DNA Master SYBR Green I kit (Roche Diagnostics). The VDCC subunit gene levels are normalized to b-actin to account for different efficiencies in cDNA synthesis. Standard errors are calculated using a propagation of error formula (Miller and Miller, 2000; Statistics and Chemometrics for Analytical Chemistry. Published by Prentice Hall (Harlow). Data from experimental and control samples are compared statistically using Kruskal-Wallis ANOVA with Dunn's multiple comparisons post-test.

The sham and contra mRNA levels are compared to the neuropathic pain model mRNA levels for each gene. It is apparent that the Cav2.2 (α1B), α2δ1, β1 and γ4 subunits show an increased level of expression in the Seltzer, axotomy, and CCl samples compared to sham and contra (Table 2). The other genes encoding for the other types of VDCC channel subunits show lower levels of gene regulation. The subunits that are consistently regulated in the Seltzer, CCl and axotomy models are then further tested in the Chung model samples.

Table 2 shows a summary of the results:

| Gene | Seltzer | CCI | Axotomy | Chung |
|---|---|---|---|---|
| α2δ1 | Increased | Increased | Increased | increased in |
| | | | | ligated and non- |
| | | | | ligated neurons |
| α2δ2 | Not consistently | Not consistently | Not consistently | Not determined |
| | regulated | regulated | regulated | |
| α2δ3 | Not consistently | Not consistently | Not consistently | Not determined |
| | regulated | regulated | regulated | |
| β1 | Increased | Increased | Not changed | Increased |
| | | | | in ligated neurons |
| β3 | Not consistently | Not consistently | Not consistently | Not determined |
| | regulated | regulated | regulated | |
| β4 | Not consistently | Not consistently | Not consistently | Not determined |
| | regulated | regulated | regulated | |
| γ2 | Not consistently | Not consistently | Not consistently | Not determined |
| | regulated | regulated | regulated | |
| γ3 | Not consistently | Not consistently | Not consistently | Not determined |
| | regulated | regulated | regulated | |
| γ4 | Increased | Increased | Not changed | Increased |
| | | | | in injured and |
| | | | | uninjured |
| | | | | neurons |
| γ7 | Not consistently | Not consistently | Not consistently | Not determined |
| | regulated | regulated | regulated | |
| γ8 | Not consistently | Not consistently | Not consistently | Not determined |
| | regulated | regulated | regulated | |
| Caᵥ2.2 (α1B) | Increased | Decreased | Not changed | Increased in |
| | | | | injured neurons |

### Example 2

### Screening Methods

Compounds that modulate Pain VDCC activity are screened for their ability to modulate calcium flow through the Pain VDCC, e.g. by measuring the change of intracellular calcium levels by determining ⁴⁵Ca-uptake or by a fluorometric determination of intracellular calcium with a calcium sensitive dye (Fluorescence assay). This is demonstrated e.g. with the following 2 screening assays.

Calcium uptake assay: Cultures of Chinese Hamster Ovary (ICN Pharmaceuticals Ltd., Basingstoke, Hampshire, U.K.) cells (therein referred to as recombinant cell) expressing human N-type voltage-dependent calcium channel (VDCC) consisting of the Caᵥ2.2 (α1B), α2δ1, β1 and γ4 subunits are prepared using multiple cassettes in tandem, e.g. clone the cDNA for two subunits in tandem with IRES (Chappell SA et al, PNAS, 97: 1536-1541), repeat with the cDNA for the other two subunits, clone both pairs into two multiple cloning site regions in a pBudCE4 vector (Invitrogen, California, U.S.) and transfect into the cell line. In addition, the cells are transfected with a potassium channel (e.g. Kir2.1 channel, Genbank Accession number AF011904) using standard protocols [McIntyre et al., J. British Journal of Pharmacology 132: 1084-1094 (2001)]. The following primers are used to amplify the coding sequence of human Kir 2.1 by RT-PCR from total RNA samples prepared from human blood eosinophils (DD10Forward: ATG GGC AGT GTG CGA ACC MC CGC TAC (SEQ ID NO: 10); DD10Reverse: TCA GTC ATA TCT CCG ATC CTC GCC GTA (SEQ ID NO: 11)). PCR products are cloned into the pCR4-TOPO vector (Invitrogen, Carlsbad, Calif.) and sequenced according to conventional methods). Cells are plated at a density of 25000 per well on 96 well plates cultured at 37° C. in 5% CO₂ in MEM medium overnight. On the day of the assay, the cells are washed four times with calcium/magnesium free HBSS plus 10 mM HEPES, pH7.4. All steps are carried out at RT. After washing the wells contain approximately 50 μl of buffer. The compound to be tested is added in 25 μl of buffer. The calcium channel is activated by eliciting depolarisation of the membrane by increasing the extracellular potassium concentration to −80 mM in Ca²⁺/Mg²⁺ free buffer containing 370 KBq of ⁴⁵Ca²⁺/ml. For negative control, potassium is omitted. Samples are incubated at RT for 15 min, then washed five times with HBSS/10 mM HEPES pH 7.4. The remaining buffer is removed from the wells and replaced with 25 μl of 0.3% SDS. After about 10 min, 200 μl of Microscint 40 scintillant is added and samples are counted on a Packard Topcount.

Compounds of interest effectively block Ca²⁺-uptake (=IC₅₀) in the range from 1 nM to 10 μM. Alternatively, the ability to block Ca²⁺-uptake in a cell expressing Pain VDCC is compared with a cell not expressing Pain VDCC.

Fluorescence assay: Cultures of Chinese Hamster Ovary (CHO) cells expressing the human N-type voltage-dependent calcium channel (VDCC) consisting of the Caᵥ2.2 (α1B), α2δ1, β1 and γ4 subunits are prepared using multiple cassettes in tandem (other method, same as above). In addition, the cells are transfected with a potassium channel (Kir2.1 channel).

The activity of test compounds are investigated using a fluorescence assay utilizing calcium sensitive dyes to measure intracellular changes of [Ca²]i. The cells are plated at a density of 25,000 per well on 96 well Costar black, clear bottomed plates cultured at 37° C. in 5% CO₂ in MEM medium overnight. On the day of the assay, cells are incubated in either 2 μM fura-2/AM or 2 μM fura-6F (Molecular Probes) made up in assay buffer [Hank's Balanced Salt Solution (HBSS, Invitrogen) containing 10 mM N-2-(hydroxyethylpiperazine-N′-[2-ethanesulfonic acid) (HEPES), pH 7.4] containing 0.01% pluronic F-127 for 30 min at room temperature. After washing twice with assay buffer 100 μl assay buffer containing the compound to be tested (range from 1 nM to 10 μM final) or assay buffer alone where appropriate, are added to each well and the plate placed in a Molecular Devices Flexstation. The fluorescence is measured over 1 min at 4s intervals using excitation wavelengths of 340 and 380 nm and emission of 520 nm. The N type calcium channels are activated by adding 20 μl of 480 mM KCl in HBSS to increase the extracellular potassium concentration to elicit a membrane depolarisation. The ratio of fluorescence intensities following excitation at 340 and 380 nm is calculated for each time point. The potassium-evoked response is calculated as the mean of the ratios in the four time-points following stimulation minus the basal ratio. Compounds of interest effectively reduce the ratio of the fluorescence intensity following excitation at 340 and 380 nm (=IC₅₀) in the range from 1 nM to 10 μM. Alternatively, the ability to reduce the ratio of the fluorescence intensity in a cell expressing Pain VDCC can be compared with a cell not expressing Pain VDCC.

### Example 3

### Antisense Oligonucleotides to β1 and γ4

Synthesis of Antisense Oligosnucleotides: Antisense oligonucleotides (ASOs) useful to inhibit gene expression, including the expression of β1 and γ4, may be made according to conventional methods. For example, ASOs against β1 or γ4 may be fully or partially phosphorothioated or fully or partially phosphodiester-18-mers with nucleotides at both ends modified with MOE (methoxy ethoxy) groups. These may be synthesized using phosphoramidite chemistry, HPLC-purified and characterized by electrospray mass spectrometry and capillary gel electrophoresis according to conventional methods. ASOs, each with a GC content between 38 and 72%, may be selected and synthesized complementary to parts of the coding region of, for example, rat or human β1 and γ4. For mismatch-containing control oligonucleotides, the approximate base composition of the match oligonucleotides may be maintained. Additionally, two control ASOs may be selected, e.g., one for rat GAPDH coding regions and a second random synthetic ASO. The format of the anti-rat-GAPDH oligonucleotide may be the same as for anti-β1 or γ4 oligonucleotides; the synthetic oligonucleotide may have its MOE ribonucleotide modifications at both ends of the sequence with phosphorothioate or phosphodiester DNA residues in the middle.

In vitro selection of β1 orγ4 ASOS: Using methods familiar to one of skill in the art, optimal ASOs may be selected from a collection of ASO candidates in vitro in order to identify the most active ASO for subsequent analyses (e.g. in vivo target validation). Such ASO candidates may be tested in comparison with mismatched ASOs (i.e. otherwise identical ASOs bearing conservative inactivating mutations), vehicle, and/or untreated controls. Once an optimal candidate ASO sequence has been identified as a target for antisense, chemical derivatives and formats, more suited for in vivo applications, and based on the identical optimal target sequence, may then be synthesized and subsequently administered in vivo.

Transfection protocol: Transfection of ASOs may be performed according to methods familiar to one of skill in the art. For example, twenty four hours before transfection, 2×10⁵ cells e.g., Chinese Hamster Ovary cells (ICN Pharmaceuticals Ltd., Basingstoke, Hampshire, U.K.) in a volume of 2 ml per well (F12 Nutrient mix (DMEM), 100 unit/millilitre Penicillin, 100 micrograms per millilitre streptomycin, 2 millimolar L-Glutamine, 10% fetal bovine serum (GIBCO-BRL, Rockville, Md.)) may be plated into 6-well plates and cultured in 5% CO₂ to yield 70-80% confluency. On the day of transfecton, a 2 fold stock transfection solution may then be prepared by diluting Lipofectin™ into serum-free OptiMEM (GIBCO-BRL, Rockville, Md.) (3 microliters Lipofectin™ per 100 nM desired final oligonucleotide concentration into 1 ml OptiMEM) and incubating for 15 minutes at room temperature. This solution may then be combined 1:1 with a 2 fold ASO-solution containing twice the desired final amount of ASO in OptiMEM. After incubating the transfection mixture for 15 minutes at room temperature to form the transfection complex, 2 ml may then be added to each of the previously aspirated well of cells. A Lipofectin™ reagent-only control and a normal cell control (untreated) may also be included. After incubation for 4 hours at 37° C., 500 microlitres of 50% FBS in MEM (Invitrogen, Carlsbad, Calif.) may then be added to each well to obtain a final FBS concentration of 10%. The cultures may then be incubated at 37° C. in a humidified incubator with 5% CO₂ for 24 hours for mRNA harvest or 48 hours for protein harvest and electrophysiology.

Real-time quantitative PCR mRNA analysis may be performed according to methods standard in the art. For example, total RNA may be isolated with the RNeasy 96 Kit (Qiagen, GmBH, Germany) according to the manufacturer's protocol. The RNA samples may be individually diluted to 1 ng/L. Five nanograms of RNA for each sample may then be mixed with gene-specific detection primers (easily determined by one of skill in the art) and with the appropriate reagents from the real-time quantitative PCR reaction kit PLATINUM™ Quantitative RT-PCR THERMOSCRIPT™ One-Step System (Gibco-BRL, Rockville, Md.) and run according to manufacturer's protocol. The rat β1 or γ4 primers with the appropriate sequences may be purchased from PE Applied Biosystems, (Foster City, Calif.). GAPDH may be chosen as a control gene for comparisons. The same RNA samples may be run with rat GAPDH primers from the TaqMan® Rodent GAPDH Control Reagents Kit (PE Applied Biosystems, Foster City, Calif.). The sequence-specific fluorescent emission signal may be detected using the ABI PRISM™ 7700 Sequence Detector (PE Applied Biosystems, Foster City, Calif.). Along with the samples, a standard from dilutions of pure template mRNA may be run to obtain absolute concentrations per inserted amount of total RNA.

β1 or γ4 RNA analysis in vitro: dose response vs. mismatch: β1 or γ4 specific ASOs may be synthesized, along with mismatch controls each bearing mutations compared with the original match ASOs. Briefly, for example, a fully phosphodiester 18-mer with 5 nucleotides at the 3′ and 5′-ends modified with 2′-O-(2-methoxyethyl) (MOE) groups, may be synthesized using phosphoramidite chemistry (Martin, P and Natt, F. EP 99-119768, US 98-168447, CAN 132:279477, AN 2000:240734) HPLC-purified, and characterised by electrospray mass spectrometry and capillary gel electrophoresis. The ASO that are most efficient at inhibiting β1 or γ4 mRNA levels, as determined by real time quantitative PCR in an in vitro assay performed on a cell line(s) that expresses relatively high levels of endogenous β1 or γ4 mRNA may then be determined. Subsequently, these may be tested against the appropriate mismatch controls, in a dose response experiment again in the cell line(s).

In vivo assay: Based on the dose response data generated in vitro, a fully or partially phosphodiester version of the ASO and missense oligonucleotide (MSO) 18-mer with 5 nucleotides at the 3′ and 5′-ends modified with 2′-O-(2-methoxyethyl) (MOE) groups, for example, may be used in vivo. ASO, MSO or vehicle may then be delivered to rats (e.g. Wistar) for up to 7 days at a desired concentration to allow cell bodies within the spinal cord and the dorsal root ganglia to take up the oligonucleotides or vehicle. ASO and MSO may be administered intrathecally via an indwelling cannula, inserted 24 h prior to or 14 days following sciatic nerve ligation, or 24 h prior to CFA injection. Rats may be anaesthetised and an incision made in the dorsal skin just lateral to the midline and approximately 10 mm caudal to the ventral iliac spines. A sterile catheter (polyethylene PE10 tubing) may be inserted via a guide cannula (20 gauge needle) and advanced 3 cm cranially in the intrathecal space approximately to the L1 level. The catheter may then be connected to an osmotic mini-pump (Alza corporation, Palo Alto, Calif.) delivering ASO, MSO or saline (1 μl/h, 7 days) which may be inserted subcutaneously in the left or right flank. The incision may then be closed with wound clips and dusted with antibiotic powder. Preliminary experiments may then be carried out to establish maximal tolerated dose. Mechanical hyperalgesia, allodynia may be measured in the following way to assess the effect of β1 or γ4 antisense oligonucleotides in reversal of hyperalgesia. Mechanical hyperalgesia may be assessed by measuring paw withdrawal thresholds of both hindpaws to an increasing pressure stimulus using an Analgesymeter (Ugo-Basile, Milan). The cut-off may be set at 250 g and the end-point taken as paw withdrawal, vocalisation or overt struggling. Paw withdrawal at pressure stimuli below 65 g is not observed after surgery. Mechanical allodynia may be assessed by measuring withdrawal thresholds to non-noxious mechanical stimuli applied with custom made von Frey hairs to the plantar surface of both hindpaws. Animals may be placed individually into wire-mesh bottom cages, with groups of 6 tested concurrently, and allowed to acclimatize for approximately 30 min. Von Frey hairs may then tested in ascending order of force with a single trial of up to 6 s for each hair until a withdrawal response was established, with a 20.6 g cut-off. This may be confirmed as the withdrawal threshold by testing a lack of response to hair with the next lowest force. Each animal may be tested only once, in random order. The statistical significance of mechanical hyperalgesia and allodynia data may be obtained from the different experimental animal groups analysed using ANOVA followed by Tukey's HSD test.

1. A method of screening for a compound comprising the steps of
a) contacting a recombinant cell with a test compound, wherein said recombinant cell expresses at least an N-type voltage-dependent calcium channel consisting of the Ca-hd v2.2 (α1B), α2δ1, β1 and γ4 subunits, provided that said cell does not express a functional calcium channel; and
b) determining the ability of said compound to modulate the calcium flow into said cell.

2. The method of claim 1, wherein said recombinant cell also expresses a potassium channel.

3. The method of claim 1, where the ability of said test compound is further compared with the ability of said test compound to modulate the calcium flow into said cell in a cell not comprising an N-type voltage-dependent calcium channel consisting of the Caᵥ2.2 (α1B), α2δ1, β1 and γ4 subunits.

4. A method for identifying a compound useful for the treatment of chronic neuropathic pain, the method comprising:
a) contacting a ligand of an N-type voltage-dependent calcium channel consisting of the Caᵥ2.2 (α1B), α2δ1, β1 and γ4 subunits with an N-type voltage-dependent calcium channel consisting of the Caᵥ2.2 (α1B), α2δ1, β1 and γ4 subunits in the presence and absence of a test compound; and
b) determining whether the test compound alters the binding of the ligand to the N-type voltage-dependent calcium channel consisting of the Caᵥ2.2 (α1B), α2δ1, β1 and γ4 subunits to an N-type voltage-dependent calcium channel consisting of the Caᵥ2.2 (α1B), α2δ1, β1 and γ4 subunits.

5. The method of claim 4, said method further comprising the steps of:
c) adding a compound identified that alters binding of the ligand to the N-type voltage-dependent calcium channel consisting of the Caᵥ2.2 (α1B), α2δ1, β1 and γ4 subunits to an N-type type voltage-dependent calcium channel consisting of the Caᵥ2.2 (α1B), α2δ1, β1 and γ4 subunits in step (b);
d) determining whether the compound alleviates chronic neuropathic pain; and
e) identifying a compound that alleviates chronic neuropathic pain in step (d) as a compound useful for the treatment of chronic neuropathic pain.

6. The method according to claim 1, wherein said Caᵥ2.2 (α1B), α2δ1, β1 and γ4 subunits consist of:
a) SEQ ID NO: 8 or 9 or variants thereof with 80% sequence identity to SEQ ID NO: 8 or 9 (for Caᵥ2.2 (α1B));
b) SEQ ID NO: 6 or 7 or variants thereof with 80% sequence identity to SEQ ID NO: 6 or 7 (for α2δ1);
c) SEQ ID NO: 1 or 2 or variants thereof with 80% sequence identity to SEQ ID NO: 1 or 2 (for β1); and
d) SEQ ID NO: 3, 4 or 5 or variants thereof with 80% sequence identity to SEQ ID NO: 3, 4 or 5 (for γ4).

7. The method according to claim 1, wherein said test compound is an antisense nucleotide sequence.

8. A purified antibody or a fragment thereof which specifically binds to the Pain voltage-dependent calcium channel (VDCC); said Pain VDCC consisting of the Caᵥ2.2 (α1B), β2δ1, β1 and γ4 subunits.

9. The antibody of claim 8, wherein said Caᵥ2.2 (α1B), α2δ1, β1 and γ4 subunits consist of:
a) SEQ ID NO: 8 or 9 or variants thereof with 80% sequence identity to SEQ ID NO: 8 or 9 (for Caᵥ2.2 (α1B));
b) SEQ ID NO: 6 or 7 or variants thereof with 80% sequence identity to SEQ ID NO: 6 or 7 (for α2δ1);
c) SEQ ID NO: 1 or 2 or variants thereof with 80% sequence identity to SEQ ID NO: 1 or 2 (for β1); and
d) SEQ ID NO: 3, 4 or 5 or variants thereof with 80% sequence identity to SEQ ID NO: 3, 4 or 5 (for γ4).

10. The antibody fragment of claim 8, which is an Fab or F(ab′)₂ fragment.

11. The antibody of claim 8, which is a polyclonal, monoclonal, chimeric or single chain antibody.

12. The method according to claim 1, wherein said test compound is an antibody of claim 6.

13. The use of an antibody according to claim 8, in medicine.

14. The use of an antibody according to claim 8, for the manufacture of a medicament for the treatment of chronic neuropathic pain.

15. The use of a compound that binds to the β1 and γ4 subunits of an N-type voltage-dependent calcium channel consisting of the Caᵥ2.2 (α1B), α2δ1, β1 and γ4 subunits for the manufacture of a medicament for the treatment of chronic neuropathic pain.

16. A method for the treatment of chronic neuropathic pain which comprises administering an effective amount of a compound that binds to the β1 and γ4 subunits of an N-type voltage-dependent calcium channel consisting of the Caᵥ2.2 (α1B), α2δ1, β1 and γ4 subunits to a patient in need of such treatment.

17. A pharmaceutical composition for the treatment of chronic neuropathic pain, which comprises a compound that binds to the β1 and γ4 subunits of an N-type voltage-dependent calcium channel consisting of the Caᵥ2.2 (α1B), α2δ1, β1 and γ4 subunits, and a pharmaceutically acceptable carrier or diluent.


SEQUENCE LISTING (62 sequences)

SEQ ID NO: 1 - protein, 596 aa, Rattus norvegicus
  PEPTIDE (1)..(596)
MVQKSGMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQTLRQNRLSSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSRTPPNPLLNRTMATAALAVSPAPVSNLQGPYLVSGDQPLDRATGEHASVHEYPGELGQPPGLYPSNHPPGRAGTLWALSRQDTFDADTPGSRNSVYTEPGDSCVDMETDPSEGPGPGDPAGGGTPPARQGSWEEEEDYEEEMTDNRNRGRNKARYCAEGGGPVLGRNKNELEGWGQGVYI

SEQ ID NO: 2 - protein, 478 aa, Homo sapiens
  PEPTIDE (1)..(478)
MVQKTSMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQKLRQNRLGSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALRRSPAPVSNLQVQVLTSLRRNLGFWGGLESSQRGSVVPQEQEHAM

SEQ ID NO: 3 - protein, 327 aa, Rattus norvegicus
  PEPTIDE (1)..(327)
MVRCDRGLQMLLTTAGAFAAFSLMAIAIGTDYWLYSSAHICNGTNLTMDDGPPPRRARGDLTHSGLWRVCCIEGIYKGHCFRINHFPEDNDYDHDSSEYLLRIVRASSVFPILSTILLLLGGLCIGAGRIYSRKNNIVLSAGILFVAAGLSNIIGIIVYISSNTGDPSDKRDEDKKNHYNYGWSFYFGALSFIVAETVGVLAVNIYIEKNKELRFKTKREFLKASSSSPYSRMPSYRYRRRRSRSSSRSTEASPSRDASPVGLKITGAIPMGELSMYTLSREPLKVTTAASYSPDQDAGFLQMHDFFQQDLKEGFHVSMLNRRTTPV

SEQ ID NO: 4 - protein, 327 aa, Homo sapiens
  PEPTIDE (1)..(327)
MVRCDRGLQMLLTTAGAFAAFSLMAIAIGTDYWLYSSAHICNGTNLTMDDGPPPRRARGDLTHSGLWRVCCIEGIYKGHCFRINHFPEDNDYDHDSSEYLLRIVRASSVFPILSTILLLLGGLCIGAGRIYSRKNNIVLSAGILFVAAGLSNIIGIIVYISSNTGDPSDKRDEDKKNHYNYGWSFYFGALSFIVAETVGVLAVNIYIEKNKELRFKTKREFLKASSSSPYARMPSYRYRRRRSRSSSRSTEASPSRDVSPMGLKITGAIPMGELSMYTLSREPLKVTTAASYSPDQEASFLQVHDFFQQDLKEGFHVSMLNRRTTPV

SEQ ID NO: 5 - protein, 327 aa, Mus musculus
  PEPTIDE (1)..(327)
MVRCDRGLQMLLTTAGAFAAFSLMAIAIGTDYWLYSSAHICNGTNLTMDDGPPPRRARGDLTHSGLWRVCCIEGIYRGHCFRINHFPEDNDYDHDSSEYLLRIVRASSVFPILSTILLLLGGLCIGAGRIYSRKNNIVLSAGILFVAAGLSNIIGIIVYISSNTGDPSDKRDEDKKNHYNYGWSFYFGALSFIVAETVGVLAVNIYIEKNKELRFKTKREFLKASSSSPYARMPSYRYRRRRSRSSSRSTEASPSRDASPVGLKITGAIPMGELSMYTLSREPLKVTTAASYSPDQDAGFLQMHDFFQQDLKEGFHVSMLNRRTTPV

SEQ ID NO: 6 - protein, 1091 aa, Rattus norvegicus
  PEPTIDE (1)..(1091)
MAAGCLLALTLTLFQSWLIGPSSEEPFPSPVTIKSWVDKMQEDLVTLAKTASGVTQLADIYEKYQDLYTVEPNNARQLVEIAARDIEKLLSNRSKALVRLAMEAEKVQAAHQWREDFASNEVVYYNAKDDLDPERNESESGSQRIKPVFIEDANFGRQISYQHAAVHIPTDIYEGSTIVLNELNWTSALDEVFKRNRDEDPTLLWQVFAADRLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFTFAFEQLLNYNVSRANCNKIIMLFTDGGEERAQEIFAKYNKDKKVRVFTFSVGQHNYDRGPIQWMACENKGYYYEIPSIGAIRINTQEYLDVLGRPMVLAGDKAKQVQWTNVYLDALELGLVITGTLPVFNVTGQSENKTNLKNQLILGVMGVDVSLEDIKRLTPRFTLCPNGYYFAIDPNGYVLLHPNLQPKNPKSQEPVTLDFLDAELENDIKVEIRNKMIDGESGEKTFRTLVKSQDERYIDKGNRTYTWTPVNGTDYRYLALVLPTYSFYYIKAKIEETITQARSKKGKMKDSETLKPDNFEESGYTFIAPREYCNDLKPSDNNTEFLLNFNEFIDRKTPNNPSCNTDLINRILLDAGFTNELVQNYWSKQKNIKGVKARFVVTDGGITRVYPKEAGENWQENPETYEDSFYKRSLDNDNYVFTAPYFNKSGPGAYESGIMVSKAVELYIQGKLLKPAVVGIKIDVNSWIENFTKTSIRDPCAGPVCDCKRNSDVMDCVILDDGGFLLMANHDDYTNQIGRFFGEIDPRMMRHLVNISLYAFNKSYDYQSVCDPGAAPKQGAGHRSAYVPSITDILQIGWWATAAAWSILQQLLLSLTFPRLLEAVEMEEDDFTASLSKQSCITEQTQYFFKNDTKSFSGLLDCGNCSRIFHVEKLMNTNLVFIMVESKGTCPCDTRLLMQAEQTSDGPDPCDMVKQPRYRKGPDVCFDNNVLEDYTDCGGVSGLNPSLWSIFGLQFILLWLVSGSRHYLW

SEQ ID NO: 7 - protein, 533 aa, Homo sapiens
  PEPTIDE (1)..(533)
MIDGESGEKTFRTLVKSQDERYIDKGNRTYTWTPVNGTDYSLALVLPTYSFYYIKAKLEETITQARSKKGKMKDSETLKPDNFEESGYTFIAPRDYCNDLKISDNNTEFLLNFNEFIDRKTPNNPSCNADLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSVADILQIGWWATAAAWSILQQFLLSLTFPRLLEAVEMEDDDFTASLSKQSCITEQTQYFFDNDSKSFSGVLDCGNCSRIFHGEKLMNTNLIFIMVESKGTCPCDTRLLIQAEQTSDGPNPCDMVKQPRYRKGPDVCFDNNVLEDYTDCGGVSGLNPSLWYIIGIQFLLLWLVSGSTHRLL

SEQ ID NO: 8 - protein, 2333 aa, Rattus norvegicus
  PEPTIDE (1)..(2333)
MVRFGDELGGRYGGTGGGERARGGGAGGAGGPGQGGLPPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDAEPVGDFPCGKEAPARLCDSDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDKNAEEKSPLDVLKRAATKKSRNDLIHAEEGEDRFVDLCAAGSPFARASLKSGKTESSSYFRRKEKMFRFLIRRMVKAQSFYWVVLCVVALNTLCVAMVHYNQPQRLTTALYFAEFVFLGLFLTEMSLKMYGLGPRSYFRSSFNCFDFGVIVGSIFEVVWAAIKPGTSFGISVLRALRLLRIFKVTKYWNSLRNLVVSLLNSMKSIISLLFLLFLFIVVFALLGMQLFGGQFNFQDETPTTNFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRYASTRHVRPDMKTHMDRPLVVEPGRDGLRGPAGNKSKPEGTEATEGADPPRRHHRHRDRDKTSASTPAGGEQDRTDCPKAESTETGAREERARPRRSHSKEAPGADTQVRCERSRRHHRRGSPEEATEREPRRHRAHRHAQDSSKEGKEGTAPVLVPKGERRARHRGPRTGPRETENSEEPTRRHRAKHKVPPTLEPPEREVAEKESNVVEGDKETRNHQPKEPRCDLEAIAVTGVGSLHMLPSTCLQKVDEQPEDADNQRNVTRMGSQPSDPSTTVHVPVTLTGPPGEATVVPSANTDLEGQAEGKKEAEADDVLRRGPRPIVPYSSMFCLSPTNLLRRFCHYIVTMRYFEMVILVVIALSSIALAAEDPVRTDSFRNNALKYMDYIFTGVFTFEMVIKMIDLGLLLHPGAYFRDLWNILDFIVVSGALVAFAFSGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGFRMELSIFYVVYFVVFPFFFVNIFVALIIITFQEQGDKVMSECSLEKNERACIDFAISAKPLTRYMPQNKQSFQYKTWTFVVSPPFEYFIMAMIALNTVVLMMKFYDAPYEYELMLKCLNIVFTSMFSLECILKIIAFGVLNYFRDAWNVFDFVTVLGSITDILVTEIAETNNFINLSFLRLFRAARLIKLLRQGYTIRILLWTFVQSFKALPYVCLLIAMLFFIYAIIGMQVFGNIALDDGTSINRHNNFRTFLQALMLLFRSATGEAWHEIMLSCLGNRACDPHANASECGSDFAYFYFVSFIFLCSFLMLNLFVAVIMDNFEYLTRDSSILGPHHLDEFIRVWAEYDPAACGRISYNDMFEMLKHMSPPLGLGKKCPARVAYKRLVRMNMPISNEDMTVHFTSTLMALIRTALEIKLAPAGTKQHQCDAELRKEISSVWANLPQKTLDLLVPPHKPDEMTVGKVYAALMIFDFYKQNKTTRDQTHQAPGGLSQMGPVSLFHPLKATLEQTQPAVLRGARVFLRQKSATSLSNGGAIQTQESGIKESLSWGTQRTQDVLYEARAPLERGHSAEIPVGQPGALAVDVQMQNMTLRGPDGEPQPGLESQGRAASMPRLAAETQPAPNASPMKRSISTLAPRPHGTQLCNTVLDRPPPSQVSHHHHHRCHRRRDKKQRSLEKGPSLSVDTEGAPSTAAGSGLPHGEGSTGCRRERKQERGRSQERRQPSSSSSEKQRFYSCDRFGSREPPQPKPSLSSHPISPTAALEPGPHPQGSGSVNGSPLMSTSGASTPGRGGRRQLPQTPLTPRPSITYKTANSSPVHFAEGQSGLPAFSPGRLSRGLSEHNALLQKEPLSQPLASGSRIGSDPYLGQRLDSEASAHNLPEDTLTFEEAVATNSGRSSRTSYVSSLTSQSHPLRRVPNGYHCTLGLSTGVRARHSYHHPDQDHWC

SEQ ID NO: 9 - protein, 2339 aa, Homo sapiens [sequence omitted]

SEQ ID NO: 10 - DNA, 27 bp, Artificial Sequence
  primer_bind (1)..(27): primers are used to amplify the coding sequence of human Kir 2.1
atgggcagtgtgcgaaccaaccgctac

SEQ ID NO: 11 - DNA, 27 bp, Artificial Sequence
  primer_bind (1)..(27): primers are used to amplify the coding sequence of human Kir 2.1
tcagtcatatctccgatcctcgccgta

SEQ ID NO: 12 - DNA, 20 bp, Artificial Sequence
  primer_bind (1)..(20): Artificial Sequence
cagacatactccagattggc

SEQ ID NO: 13 - DNA, 20 bp, Artificial Sequence
  primer_bind (1)..(20): Artificial Sequence
tagtgtctgctgccagatac

SEQ ID NO: 14 - DNA, 20 bp, Artificial Sequence
  primer_bind (1)..(20): Artificial Sequence
cagcctggataaccatggtt

SEQ ID NO: 15 - DNA, 20 bp, Artificial Sequence
  primer_bind (1)..(20): Artificial Sequence
agctgtgatagatgagacca

SEQ ID NO: 16 - DNA, 20 bp, Artificial Sequence
  primer_bind (1)..(20): Artificial Sequence
aacaaggagagcagtgacag

SEQ ID NO: 17 - DNA, 20 bp, Artificial Sequence
  primer_bind (1)..(20): Artificial Sequence
agaagagactcgaaaccagg

SEQ ID NO: 18 - DNA, 20 bp, Artificial Sequence
  primer_bind (1)..(20): Artificial Sequence
acatgatgcagaaggcgttg

SEQ ID NO: 19 - DNA, 20 bp, Artificial Sequence
  primer_bind (1)..(20): Artificial Sequence
ccagctcattcttattgcgc

SEQ ID NO: 20 - DNA, 20 bp, Artificial Sequence
  primer_bind (1)..(20): Artificial Sequence
cttacgatgtggtaccatcc

SEQ ID NO: 21 - DNA, 20 bp, Artificial Sequence
  primer_bind (1)..(20): Artificial Sequence
tgcactcattgtggtcttgg

SEQ ID NO: 22 - DNA, 20 bp, Artificial Sequence
  primer_bind (1)..(20): Artificial Sequence
acagacatgatgcagaaggc

SEQ ID NO: 23 - DNA, 20 bp, Artificial Sequence
  primer_bind (1)..(20): Artificial Sequence
atctaactccaacctgcctg

SEQ ID NO: 24 - DNA, 20 bp, Artificial Sequence
  primer_bind (1)..(20): Artificial Sequence
tgattggtggataggaaggc

SEQ ID NO: 25 - DNA, 20 bp, Artificial Sequence
  primer_bind (1)..(20): Artificial Sequence
aatcttcttccaccagaggg

SEQ ID NO: 26 - DNA, 20 bp, Artificial Sequence
  primer_bind (1)..(20): Artificial Sequence
caggagatgttccagaagac

SEQ ID NO: 27 - DNA, 20 bp, Artificial Sequence
  primer_bind (1)..(20): Artificial Sequence
tggtcatgctcagatctgtc

SEQ ID NO: 28 - DNA, 20 bp, Artificial Sequence
  primer_bind (1)..(20): Artificial Sequence
acctacaagacggccaattc

SEQ ID NO: 29 - DNA, 20 bp, Artificial Sequence
  primer_bind (1)..(20): Artificial Sequence
gagaggaccatatccctcta

SEQ ID NO: 30 - DNA, 20 bp, Artificial Sequence
  primer_bind (1)..(20): Artificial Sequence
gacatctctcagaagacagc

SEQ ID NO: 31 - DNA, 20 bp, Artificial Sequence
  primer_bind (1)..(20): Artificial Sequence
acaggttgctgacataggac

SEQ ID NO: 32 - DNA, 20 bp, Artificial Sequence
  primer_bind (1)..(20): Artificial Sequence
accttatgcagcagcagatc

SEQ ID NO: 33 - DNA, 20 bp, Artificial Sequence
  primer_bind (1)..(20): Artificial Sequence
caaagtcctggagctcatag

SEQ ID NO: 34 - DNA, 20 bp, Artificial Sequence
  primer_bind (1)..(20): Artificial Sequence
ttaccgagtcttccaactcc

SEQ ID NO: 35 - DNA, 20 bp, Artificial Sequence
  primer_bind (1)..(20): Artificial Sequence
gtgagagaagagcatgcatc

SEQ ID NO: 36 - DNA, 20 bp, Artificial Sequence
  primer_bind (1)..(20): Artificial Sequence
ttcaccatccagtgtctgca

SEQ ID NO: 37 - DNA, 20 bp, Artificial Sequence
  primer_bind (1)..(20): Artificial Sequence
catctgcaatctcctgcttg

SEQ ID NO: 38 - DNA, 20 bp, Artificial Sequence
  primer_bind (1)..(20): Artificial Sequence
agagaccagaagcagcttag

SEQ ID NO: 39 - DNA, 20 bp, Artificial Sequence
  primer_bind (1)..(20): Artificial Sequence
catgtctgttgggtcagaag

SEQ ID NO: 40 - DNA, 20 bp, Artificial Sequence
  primer_bind (1)..(20): Artificial Sequence
ggaacaacaacctgaccttc

SEQ ID NO: 41 - DNA, 20 bp, Artificial Sequence
  primer_bind (1)..(20): Artificial Sequence
gatcatgaagaaggagccca

SEQ ID NO: 42 - DNA, 20 bp, Artificial Sequence
  primer_bind (1)..(20): Artificial Sequence
acaggcgataactggaatgg

SEQ ID NO: 43 - DNA, 20 bp, Artificial Sequence
  primer_bind (1)..(20): Artificial Sequence
gatgaagacctgtctcttgc

SEQ ID NO: 44 - DNA, 20 bp, Artificial Sequence
  primer_bind (1)..(20): Artificial Sequence
tgaccctcttcttcatcctg

SEQ ID NO: 45 - DNA, 20 bp, Artificial Sequence
  primer_bind (1)..(20): Artificial Sequence
gatctagtgctctgactcag

SEQ ID NO: 46 - DNA, 20 bp, Artificial Sequence
  primer_bind (1)..(20): Artificial Sequence
gctgtttgatcgaggtgttc

SEQ ID NO: 47 - DNA, 20 bp, Artificial Sequence
  primer_bind (1)..(20): Artificial Sequence
tgtacatggagatctccgtg

SEQ ID NO: 48 - DNA, 20 bp, Artificial Sequence
  primer_bind (1)..(20): Artificial Sequence
atgaggatgtgtgacagagg

SEQ ID NO: 49 - DNA, 20 bp, Artificial Sequence
  primer_bind (1)..(20): Artificial Sequence
tgtggaagcctttgctgatg

SEQ ID NO: 50 - DNA, 21 bp, Artificial Sequence
  primer_bind (1)..(21): Artificial Sequence
accaacctgaccatggacgac

SEQ ID NO: 51 - DNA, 21 bp, Artificial Sequence
  primer_bind (1)..(21): Artificial Sequence
tacctgtagctcggcatcctg

SEQ ID NO: 52 - DNA, 20 bp, Artificial Sequence
  primer_bind (1)..(20): Artificial Sequence
tgagcagtgtctttgctgtc

SEQ ID NO: 53 - DNA, 20 bp, Artificial Sequence
  primer_bind (1)..(20): Artificial Sequence
atccatacttagggtgacgg

SEQ ID NO: 54 - DNA, 20 bp, Artificial Sequence
  primer_bind (1)..(20): Artificial Sequence
aagctgttgggccttaagag

SEQ ID NO: 55 - DNA, 20 bp, Artificial Sequence
  primer_bind (1)..(20): Artificial Sequence
aagtcccatcataacctcgc

SEQ ID NO: 56 - DNA, 20 bp, Artificial Sequence
  primer_bind (1)..(20): Artificial Sequence
gggtggagctcaacacctac

SEQ ID NO: 57 - DNA, 18 bp, Artificial Sequence
  primer_bind (1)..(18): Artificial Sequence
gcccagggaccaggagta

SEQ ID NO: 58 - DNA, 19 bp, Artificial Sequence
  primer_bind (1)..(19): Artificial Sequence
atacagaggcaccccaagg

SEQ ID NO: 59 - DNA, 20 bp, Artificial Sequence
  primer_bind (1)..(20): Artificial Sequence
cggtcagcactgactactgg

SEQ ID NO: 60 - DNA, 19 bp, Artificial Sequence
  primer_bind (1)..(19): Artificial Sequence
atgacctcgtcgttgatgc

SEQ ID NO: 61 - DNA, 21 bp, Artificial Sequence
  primer_bind (1)..(21): Artificial Sequence
atgaccatcgccatcatcagc

SEQ ID NO: 62 - DNA, 18 bp, Artificial Sequence
  primer_bind (1)..(18): Artificial Sequence
cgcccagaatgatgttcc